(19) United States
(12) Patent Application Publication
(10) Pub. No.: US 2022/0178833 A1
(43) Pub. Date: Jun. 9, 2022

(54) METHOD FOR DETECTING SKATOLE IN A SAMPLE OF PIG ADIPOSE TISSUE

(71) Applicant: COMMISSARIAT À L'ÉNERGIE ATOMIQUE ET AUX ÉNERGIES ALTERNATIVES, Paris (FR)

(72) Inventors: Emmanuel Scorsone, Magny-Les-Hameaux (FR); Matthieu Hamel, Cherbourg (FR)

(21) Appl. No.: 17/597,518

(22) PCT Filed: Jul. 8, 2020
(86) PCT No.: PCT/FR2020/051209

§ 371 Date: Jan. 10, 2022

(30) Foreign Application Priority Data
Jul. 12, 2019 (FR) 1907882

(51) Int. Cl. G01N 21/76; G01N 1/40; G01N 33/483

(52) U.S. Cl. CPC G01N 21/763; G01N 33/4833; G01N 1/4044

(57) ABSTRACT

A method for detecting the presence of skatole in a sample of pig adipose tissue, wherein the method comprises at least the steps of: a) subjecting an organic extract of the adipose tissue sample to an electrochemiluminescence reaction; b) measuring the luminescence intensity during step a) and, if the measured luminescence intensity exceeds a threshold value, deducing the presence of skatole in the sample of adipose tissue. The method also makes it possible, if skatole is present in the organic extract, to determine the content thereof.

### TECHNICAL FIELD

The invention relates to the agri-food sector and, in particular, to the production and distribution of pork meat.

More specifically, the invention relates to a method for detecting the presence of skatole, or 3-methylindole (3-MIH), in a sample of a pig adipose tissue and, if it is present, determining the content thereof with a very high degree of sensitivity and very high specificity with respect to other compounds which may also be present in the pig adipose tissue.

As skatole, along with androsterone, is responsible for a strong and unpleasant odour, referred to as boar taint, which is released when cooking the meat of whole male pigs, the invention is primarily suitable for use in abattoirs for sorting the carcasses of whole male pigs for the purpose of isolating those whose meat carries boar taint and directing them to a suitable processing circuit.

It is also suitable for use as a research tool, in particular for studying the factors that influence the production of skatole in the adipose tissue of whole male pigs for the purpose of developing methods which make it possible to prevent or reduce boar taint, for example by genetic selection or by modifying the conditions of animal husbandry (feed, housing conditions, composition of animal groups in terms of age, sex, etc.).

### Prior Art

Boar taint is caused mainly by an accumulation of skatole and androsterone and, to a lesser extent, indole in the adipose tissue—or fatty tissue—of whole male pigs, i.e. non-castrated pigs.

This smell, which is released when cooking the pork, is generally considered to be nauseating by consumers.

Historically, to prevent boar taint, male pigs were castrated before the age of sexual maturity.

However, for the last ten years, for ethical and animal welfare reasons but also for economic reasons a number of livestock farmers have stopped castrating male pigs with the support of the European Union.

Therefore, it is necessary to have a method for reliably detecting, in slaughter lines, the carcasses of whole male pigs whose meat carries boar taint.

Sorting carcasses by detecting skatole in the abattoir poses a number of problems.

There are many reasons for these problems.

Firstly, the detection of skatole in the fatty tissue of pigs requires an extremely sensitive detection method. Indeed, the threshold at which a consumer rejects pork meat is set at approximately 0.2 μg skatole per gram of adipose tissue. In analytical terms, this means that, in the case for example of a complete extraction of skatole present in a gram of adipose tissue and concentrated in one mL of a suitable solvent, the objective is to be able to detect a concentration threshold of skatole of 1.53 μmol/L in this mL of solution. Likewise, after heating the adipose tissue and volatilising the molecules of skatole, the concentration of skatole in the vapour phase is in the order of several tens of ppb, which does not make it easier to measure skatole in such a phase.

Another problem relates to the selectivity of the detection of skatole. Indeed, as skatole is an indole compound, which originates from the degradation of tryptophan by intestinal bacteria, it is present in pork fat along with other indolic compounds such as indole, which are likely to interfere with the detection of skatole by producing false positives.

Lastly, in addition to this there are:
time constraints, the detection of skatole must be able to be carried out on several hundreds of carcasses per day for most abattoirs;
constraints relating to the conditions inside abattoirs which are uncontrolled environments with strong variations in temperature throughout the year and high humidity levels;
cost restrictions, the detection of skatole should not have a significant impact on the final sale price of the pork;
variability in the composition of adipose tissue taken from the carcasses (water content, blood content, etc.); and
a problem with the traceability of the samples analysed in the abattoir.

Methods for detecting skatole are known.

The most common methods use high performance liquid phase chromatography or gas phase chromatography (cf., for example, K. Verplanken et al, Journal of Chromatography A 2016, 1462, 124-133). These methods are not suitable for use in an abattoir as they require long analysis times, highly qualified operators, expensive equipment and consumables and a high level of maintenance. In addition, given the complexity of the fatty matrix, they generally have an inadequate level of selectivity.

Recently, Denmark announced an automated method based on mass spectrometry after the suitable processing of the samples. However, this method is also expensive and probably poorly adapted to the conditions of use in an abattoir. There is more than one problem with the traceability of the samples which are analysed in series in the spectrometer.

Immunological methods, in particular based on fluoro-immunological dosing using europium(III) as a fluorescent marker, have been studied (cf., for example, M. P. Aguilar-Caballos et al, Analytica Chimica Acta 2002, 460, 271-277) but these studies are still marginal because of the non-availability of specific antibodies.

A method for the colorimetric detection of skatole using Ehrlich's reagent has been proposed (cf. international application WO 83/00928). This method, which is simple to use and inexpensive, was used in Danish abattoirs in the 1990s. Unfortunately, it had to be abandoned due to the lack of selectivity of the Ehrlich's reagent, in particular with regard to indole and due to the unreliability of the results.

Electrochemical methods have also been proposed for the detection of skatole (cf., for example, European patent application 2 966 441). These methods consist of measuring the oxidation current of skatole on the surface of an electrode, most often carbon (diamond or glassy carbon). These methods are very sensitive, but they suffer a major problem with selectivity, again due to the complexity of the fatty matrix.

More recently, methods using Raman spectroscopy have been proposed (cf., for example, X. Liu et al, Meat Science 2016, 116, 133-139). These methods have a global approach consisting of simultaneously analysing the androstenone and skatole in adipose tissue. However, the results presented so far highlight an unacceptable number of false positives and false negatives.

Finally, electronic noses have been developed (cf., for example, J. S. Vestergaard et al, Meat Science 2006, 74, 564-577). The functioning of these electronic noses is based on the use of a network of cross-reactive sensors providing a global “chemical fingerprint” of the sample to be analysed. Although, potentially suitable for measurements in an abattoir, the electronic noses still have a major problem with selectivity. Furthermore, the sensors used in the electronic noses are often sensitive to humidity which then becomes a major hindrance.

Due to the absence of a physico-chemical method for the detection of skatole which is at the same time sensitive, selective and suitable for use in an abattoir, the majority of abattoir lines for whole male pig currently use a panel of “human noses”. The protocol consists of heating a sample of adipose tissue using a solder iron or an equivalent device, most often directly on the carcasses, and operators specifically trained for this task then smell the odorous compounds released thereby. This method of detection is expensive and restrictive, unpleasant for the operators and is only effective when a small number of carcasses need to be sorted, as the olfactory receptors of the operators soon become saturated. Furthermore, this method produces highly variable results that depend on the operators with only a 70% rate of true positives.

In view of the above, the inventors have the objective of providing a physico-chemical method which makes it possible to detect the presence of skatole in samples of pig adipose tissue with high sensitivity and high specificity such that it leads to extremely reliable results.

They also have the objective that this method will also allow the dosing of this skatole if desired if skatole is present in the samples of adipose tissue.

They also have the objective that the implementation of this method can be performed in abattoirs, regardless of the existing temperature and humidity conditions, and that this implementation will be sufficiently simple and quick to be compatible with the time restraints of carcass sorting that abattoirs are subjected to.

They have also set themselves the aim that the implementation of this method will have a cost which does not have a significant impact on the final sale price of the pork meat.

They have also set themselves the aim that this method will permit the traceability of the samples of adipose tissue of the analysed pigs.

### DISCLOSURE OF THE INVENTION

All of these aims are achieved by the invention which firstly proposes a method for detecting the presence of skatole in a sample of a pig adipose tissue, which method is characterised in that it comprises at least the steps consisting of:

a) subjecting an organic extract of the sample of adipose tissue to an electrochemiluminescence reaction;

b) measuring the intensity of the luminescence during step a) and, if the measured luminescence intensity exceeds a predetermined threshold value, deducing the presence of skatole in the sample of adipose tissue.

Thus, the detection of skatole by the method of the invention is based on the technique of electrochemiluminescence.

It is noted that electrochemiluminescence (or ECL), also referred to as electrogenerated chemiluminescence, is a technique which consists of initiating a luminescence process by a transfer of electrons occurring directly on the surface of a working electrode.

There are different ECL mechanisms.

In the context of the invention, it is preferred that the luminescence process is initiated by the application of a cathodic potential to a working electrode immersed in the organic extract to induce the formation of superoxide ions (O₂⁻.) by reduction of the dioxygen dissolved in this extract.

In this case, if skatole is present in the organic extract, the reduction of oxygen to superoxide ions is followed by:
the formation of the conjugate base of skatole according to the reaction:

3-MIH+O₂⁻.→3-MI⁻+HO₂., then
the formation of hydroperoxide radicals according to the reaction:

O₂⁻.+HO₂.→O₂+HO₂⁻.

The subsequent application of an anodic potential to the working electrode then permits the oxidisation of the conjugate base of skatole which, once oxidised, will react with the hydroperoxide radicals to result in the formation of N-(2-acetylphenyl)-formamide in the excited state, which by deexcitation (or, in other words, by returning to its fundamental state), emits a measurable luminescence.

It should be noted that this ECL mechanism of skatole has been studied and described by T. Okajima and T. Ohsaka (cf. Journal of Electroanalytical Chemistry 2002, 523, 34-39). However, this study has been performed in a purely theoretical context, aimed at understanding the chemical processes involved in the ECL of skatole in solution in acetonitrile but without any practical application of these processes being considered and in particular an application for the detection of skatole in the adipose tissues of pigs.

In view of the above, it is preferred that the organic extract meets the following conditions:
comprises an aprotic organic solvent;
is anhydrous, i.e. comprises at most 1 wt. % water; and
is electroconductive, which is made possible by the presence of a ground salt in this extract.

To achieve this, the invention proposes the prior preparation of the organic extract by:

i) separating the fat present in the sample of adipose tissue from the non-fatty elements also present in the sample of adipose tissue, such as skin residues, muscle fibres, etc.;

ii) dehydrating the fat present in the sample of adipose tissue, knowing that pig tissue naturally comprises in the order of 15 wt. % water;

iii) dissolving the fat obtained at the end of step ii) in an aprotic organic solvent to obtain an organic solution and heating the organic solution for a sufficient period to enable the extraction of skatole if the latter is present;

iv) degreasing the organic solution obtained at the end of step iii); and

v) adding an anhydrous ground salt to the organic solution obtained at the end of step iv);

and wherein steps i) and ii) may be carried out successively or simultaneously.

According to the invention, steps i) and ii) are preferably carried out simultaneously.

To achieve this, it is possible for example to heat the sample of adipose tissue to a temperature equal to at least 100° C. and, preferably, between 100° C. and 150° C., and in an open container such that the water present in the sample of adipose tissue can escape in the form of water vapour. This makes it possible to liquefy the fat present in the sample of adipose tissue and, thus, to facilitate its subsequent separation from the non-fatty elements also present in this sample which will remain in the solid state, and at the same time to dehydrate it. This also makes it possible to denature the proteins present in the sample of adipose tissue which will then precipitate during step iii), which contributes to reducing the amount of electroactive species which are present in the organic extract.

Alternatively, it is also possible to subject the sample of adipose tissue to heating at a temperature of less than 100° C. with vacuum drawing.

The duration of the heating is selected in particular according to the mass of the sample of adipose tissue, the form, pre-cut or not, in which it is presented and if it is pre-cut, the size of the pieces of the sample, the configuration of the container in which the heating is performed, the mode of heating (with or without microwaves for example), the use or not of vacuum drawing and the temperature selected to perform the heating.

In step iii), the fat obtained at the end of the preceding step or the two preceding steps (if these are carried out simultaneously) is dissolved in an aprotic organic solvent and the resulting organic solution is heated.

In the above and in the following the term “aprotic”, when applied to an organic solvent, is taken in its accepted form, namely that it denotes an organic solvent the molecule of which is free of acidic hydrogen atom, i.e. bonded to a heteroatom such as a nitrogen, oxygen or sulphur atom.

According to the invention, the aprotic solvent is advantageously a polar aprotic solvent, i.e. having a non-zero dipolar moment, such as acetonitrile, dimethyl sulfoxide, propylene carbonate or γ-butyrolactone, preference being given to acetonitrile.

The heating of step iii) is advantageously performed, with stirring, at a temperature of 50° C. to 80° C., for 1 minute to 10 minutes, and this, in a closed container to prevent the aprotic organic solvent from evaporating.

Step iv) is performed advantageously by subjecting the organic solution obtained at the end of step iii) to centrifugation in order to break the emulsion formed during step iii) due to the stirring, then maintaining this organic solution at a temperature lower than or equal to 4° C. but greater than the solidification temperature of the aprotic organic solvent so as to fix the fatty part of the organic solution but without the organic solvent solidifying and then eliminating the fatty part that has been fixed.

The ground salt, which is added to the organic solution obtained at the end of step iv), can be chosen from a very large number of salts that must be on the one hand soluble in the aprotic organic solvent, and on the other hand chemically and electrochemically inert so as not to disturb the ECL reaction nor to induce an adverse reaction with skatole. As well known to electrochemists, this salt can be in particular a tetraalkylammonium tetrafluoroborate, hexafluorophosphate or perchlorate, wherein the alkyl group comprises 1 to 6 carbon atoms, such as tetrabutylammonium tetrafluoroborate or tetrabutylammonium hexafluorophosphate, this type of salt having in fact a remarkable stability in an organic medium.

Furthermore, the ground salt is added to the organic solution obtained at the end of step iv) in an amount such that its concentration in the organic extract is typically between 0.01 mol/L and 1 mol/L, preferably between 0.05 mol/L and 0.5 mol/L, and, more preferably equal to 0.1 mol/L.

According to the invention, it is possible to add to the organic solution obtained, either at the end of step iii), step iv) and/or step v), a drying agent such as a hygroscopic salt of the anhydrous sodium or magnesium type, or a molecular screen (3 or 4 angstroms) to complete the dehydration carried out in step ii).

In a preferred embodiment of the method of the invention, the organic extract further comprises a strong base, in which case this strong base is preferably added to the organic solution obtained at the end of step v).

The presence of a strong base in the organic extract makes it possible firstly to deprotonate the organic acids present in this extract. Indeed, as will be shown in the following, skatole becomes in several stages of the ECL reaction a proton acceptor. However, any organic extract prepared from a sample of pig adipose tissue contains a certain number of proton donor compounds, and in particular organic acids, which are essentially fatty acids soluble in organic solvents that the degreasing operations as performed in step iv) do not make it possible to eliminate. It is therefore desirable to neutralise these compounds, which is made possible by the presence of a strong base in the organic extract.

The presence of a strong base in the organic extract also makes it possible to ensure the disappearance of any trace of residual water in the organic extract.

A strong base is defined within the meaning of the invention as a base that is capable of deprotonating water.

This base can be in particular an organic base such as triethylamine, imidazole, histidine, or any other tertiary amine, an alcoholate or an amide of an alkali metal such as alcoholate or sodium amide, a hydride or an alkali metal or alkaline earth metal such as sodium hydride, lithium hydride, potassium hydride or rubidium hydride, a nitride of an alkali metal or alkaline earth metal such as sodium nitride.

Preference is given to a strong base having the highest possible pKa.

A particularly preferred strong base is sodium hydride which besides having a conjugated acid with a high pKa, has the advantage of being inexpensive, easy to obtain and for which the reaction with acid species results in their sodium equivalent—which precipitates and can therefore be eliminated—with the release in gaseous form of the hydrogen present in the organic extract. For example, the reaction of sodium hydride with water, which is potentially still present in the organic extract, is as follows:

H₂O+NaH→NaOH (which precipitates)+H₂(gas).

As known per se, the ECL reaction is preferably performed in an electrochemical cell, the terms “electrochemical cell” denoting here the assembly formed by a cuvette-type container or the like, in which the organic extract is placed for the ECL reaction, and at least two electrodes, namely a working electrode and a counter-electrode.

As indicated above, the ECL reaction which is preferred in the context of the invention comprises the application of a cathodic potential to the working electrode of the electrochemical cell to induce the formation of superoxide ions, followed by the application of an anodic potential to this same electrode to induce the oxidation of the conjugate base of skatole if the latter is present in the organic extract.

This can be achieved according to different electrochemical protocols and in particular by:
a potential sweep, in which case a sweep towards the negative potentials followed by a sweep towards the positive potentials are applied to the working electrode;
a potential step, in which case a constant negative potential is applied to the working electrode, for a period sufficient to saturate the surface of this electrode with superoxide ions, followed by a constant positive potential, also for a period sufficient to saturate the surface of the working electrode in oxidised skatole; or
by a series of alternating cathodic and anodic potential pulses.

It goes without saying that the electrochemical parameters have to be adapted in particular according to the configuration of the electrochemical cell used, the nature of the working electrode and of the counter-electrode used, namely the electrode material which constitutes these electrodes, the way in which the organic extract has been prepared and therefore its composition and viscosity. If a reference electrode or pseudo-reference electrode is used, the electrochemical parameters also have to be adapted according to the nature of this electrode.

For example, for organic extracts prepared as described above and electrochemical cells provided with a working electrode and a boron-doped diamond counter-electrode as well as a pseudo-reference electrode made of platinum, excellent results have been obtained by applying to the working electrode:
in the case of a potential sweep: a sweep from 0 V to a negative potential between −1.5 V and −2.5 V (vs Pt), for example equal to −2 V (vs Pt), then a sweep from this negative potential to a positive potential greater than or equal to +0.5 V (vs Pt), for example equal to +0.8 V (vs Pt), with a constant sweep speed between 10 mV/s and 100 mV/s, for example 50 mV/s;
in the case of a potential step: a constant negative potential less than −1.5 V (vs Pt), for example equal to −2 V (vs Pt), for 0.1 seconds to 45 seconds, then a constant positive potential greater than 0.5 V (vs Pt), for example equal to +1 V (vs Pt), for 10 seconds to 30 seconds; and
in the case of alternating cathodic and anodic potential pulses: a negative potential less than −1.5 V (vs Pt), for example equal to −2 V (vs Pt), followed by a positive potential greater than 0.5 V (vs Pt), for example equal to +1 V (vs Pt), at a frequency ranging from 0.5 hertz to 5 hertz, for example equal to 1 hertz.

The choice of the container of the electrochemical cell is not critical in itself.

However, it is desirable that this container is made from a material resistant to organic solvents, saline media and, if a strong base is used, to alkaline media.

Furthermore, it is advisable that this container is made of an optically transparent material in the luminescence emission wavelength range or that it has at least one wall made of a material with such transparency if the detection of the photons emitted is performed by an optical detector located facing one of its walls.

The choice of the electrodes of the electrolytic cell is also not critical.

If the ECL reaction is based on the formation of superoxide ions, then the working electrode can be made of any electrode material allowing the formation of such ions as carbon (graphite, glassy carbon, doped diamond, for example boron or nitrogen, etc.), a noble metal (gold, platinum, palladium, iridium, etc.) or an alloy of noble metals.

The counter-electrode can be formed by a different electrode material or the same electrode material as that forming the working electrode.

In the context of the invention, it is preferred that the working electrode and the counter-electrode are made of doped diamond, in particular doped with boron, due to the fact that this material is highly conductive, has a very high stability, a natural resistance to fouling due to the high atomic density of the diamond. This resistance to fouling is of particular interest given that the organic extract may comprise a certain number of compounds derived from pig adipose tissue, including fatty acids, which can quickly foul the surface of the electrodes. Furthermore, in the case of fouling, this type of electrode can be easily cleaned electrochemically, for example by the method described in U.S. Pat. No. 9,121,107. Lastly, this type of electrode has a large potential window which makes it possible to apply high potentials without electrolysing the solvent present in the organic extract.

If a reference electrode is used, then this can be in particular a saturated calomel electrode (SCE) or a silver chloride electrode (Ag/AgCl), optionally with a double junction, as used traditionally in electrochemistry. However, in the context of the invention, it is preferred to use a pseudo-reference electrode made of a noble metal such as platinum because this type of electrode can also be easily cleaned, for example by passing through a flame.

Advantageously, the electrochemical cell (i.e. container and electrodes) are made of low-cost materials (plastic container, carbon paste electrodes, etc.) so as to be for single use, which makes it possible, on the one hand, to overcome the problems of fouling of the electrodes and, on the other hand, to ensure the traceability of the samples of fatty tissue analysed, for example by referencing each electrochemical cell in relation to a pig carcass.

With regard to the optical detector, the latter may be a photomultiplier coupled to a bialkali, super-bialkali or ultra-bialkali photocathode, an avalanche photodiode, a silicon photocathode photomultiplier, a spectrometer and, in particular a spectrofluorometer, a CCD (Charge-Coupled Device) sensor detector, a CMOS (Complementary Metal Oxide Semiconductor) sensor detector, etc.

According to the invention, the threshold value is preferably at least equal to 150% of the average value of the intensity of the luminescence corresponding to the background noise of the optical detector.

As indicated above, the method of the invention also makes it possible, if skatole is present in the organic extract, to determine the content of the latter.

Thus, if the presence of skatole has been deduced in step b), the method further comprises advantageously a quantification of the skatole present in the organic extract by comparing the maximum luminescence intensity measured in step b) with a calibration curve, this quantification being carried out during step b) or following step b).

The method of the invention makes it possible to detect the presence of skatole in pig adipose tissue with high sensitivity since a detection limit of around 20 nmol/L skatole was able to be obtained.

The threshold for the rejection of pork meat by the consumer is set at about 0.2 μg skatole per gram of adipose tissue, or raw fat, which corresponds, with the assumption of a complete extraction of the skatole present in 1 g of adipose tissue in 1 mL of an organic solvent, to a skatole concentration of 1.53 μmol/L.

As a result, the detection limit of skatole by the method of the invention is around 3 orders of magnitude below the concentration of skatole to be detected.

Furthermore, as demonstrated by the experiments reported below, the detection of skatole by the method of the invention is highly specific since other indolic compounds present in the adipose tissues of pigs such as indole are not detected by this method.

The absence of phototonic excitation (by UV, filtered white light or other) in the electrolysis cell prevents any spontaneous fluorescence from potential interferents and thus makes it possible to obtain a very high signal/noise ratio.

Furthermore, the method of the invention is robust, both in terms of the equipment it requires and in its implementation, simple, fast and inexpensive.

It is therefore particularly well suited for use in an abattoir.

Thus, the invention also relates to a method for sorting the carcasses of whole male pigs, which is characterised in that it comprises implementing a method for detecting skatole as defined above.

Other features and advantages of the invention are given in the following description which relates to experiments, which made it possible to validate the invention, and which is given with reference to the appended figures.

It goes without saying however that this additional description is given only by way of illustration of the subject-matter of the invention and should not be interpreted as a limitation of this subject-matter.

### BRIEF DESCRIPTION OF THE FIGURES

FIG. 1 represents schematically a first electrochemical cell which has been used in the experiments described above, in cross-section.

FIG. 2 represents schematically a second electrochemical cell which has been used in the experiments described below, ¾ view.

FIG. 3 illustrates the voltammogram obtained by subjecting an organic extract of a sample of pig adipose tissue not contaminated with skatole, to which synthetic skatole has been added, to an ECL reaction by potential sweeping.

FIG. 4 illustrates the luminescence measured simultaneously with the recording of the voltammogram shown in FIG. 3.

FIG. 5 illustrates the chronoamperogram obtained by subjecting an organic extract of a sample of pig adipose tissue not contaminated with skatole, to which synthetic skatole has been added, to an ECL reaction by potential step.

FIG. 6 illustrates the luminescence measured simultaneously with the recording of the chronoamperogram shown in FIG. 5.

FIG. 7 illustrates the current response obtained by subjecting a solution comprising synthetic skatole and tetrabutylammonium hexafluorophosphate in acetonitrile to an ECL reaction by applying a repetition of alternating cathodic and anodic voltage pulses.

FIG. 8 illustrates the luminescence measured simultaneously with recording the current response shown in FIG. 7.

FIG. 9 illustrates the luminescence measured by subjecting a solution comprising synthetic skatole and tetrabutylammonium hexafluorophosphate in acetonitrile to an ECL reaction by potential sweep.

FIG. 10 illustrates the luminescence measured by subjecting a solution comprising indole and tetrabutylammonium hexafluorophosphate in the acetonitrile to an ECL reaction by potential sweep.

FIG. 11 illustrates the luminescence measured by subjecting an organic extract of a sample of pig adipose tissue contaminated by skatole to an ECL reaction by potential step.

FIG. 12 illustrates the luminescence measured by subjecting an organic extract of a sample of pig adipose tissue not contaminated with skatole to an ECL reaction by potential step.

FIG. 13 illustrates two standard curves produced by subjecting organic extracts of samples of pig adipose tissue not contaminated with skatole but to which synthetic skatole has been added in an amount of 0.1 μmol/L to 5 μmol/L (curve 1) and solutions comprising 0.1 μmol/L to 5 μmol/L synthetic skatole and tetrabutylammonium hexafluorophosphate in acetonitrile (curve 2) to an ECL reaction by potential step.

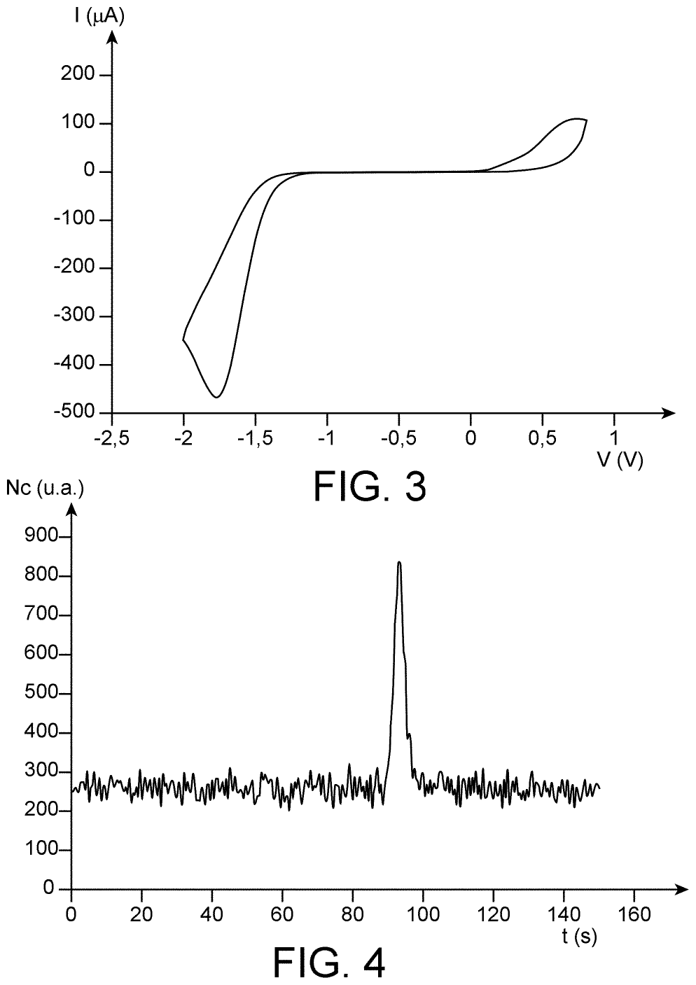

In FIG. 3, the y-axis corresponds to the intensity, denoted I and expressed in microamperes (μA), of the current measured at the working electrode, whereas the x-axis corresponds to the potential, denoted V and expressed in volts (V) in relation to the potential of the reference electrode, applied to the working electrode.

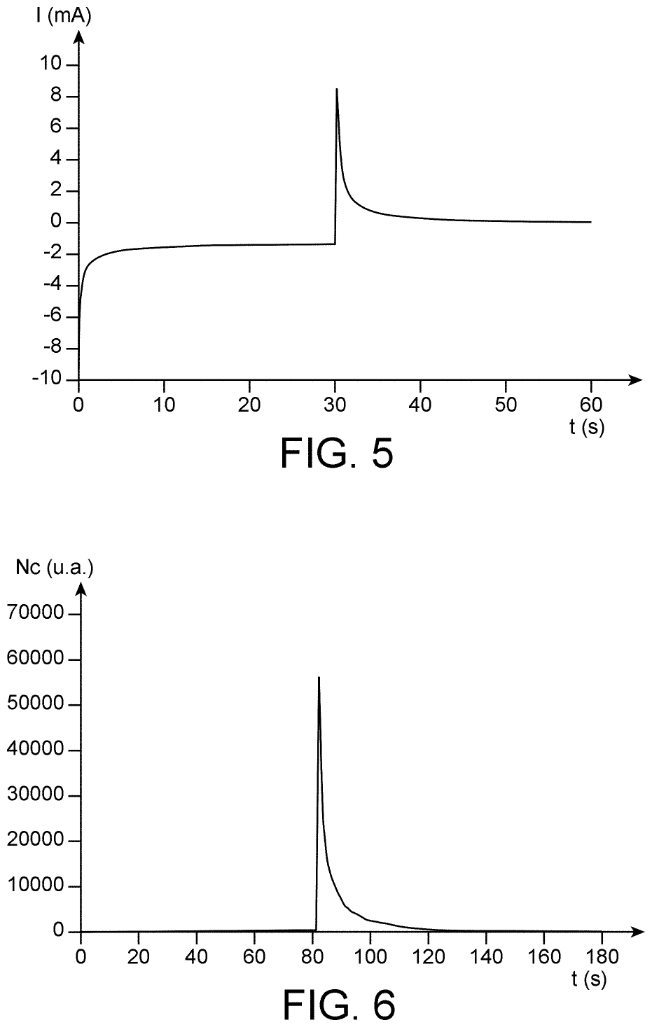

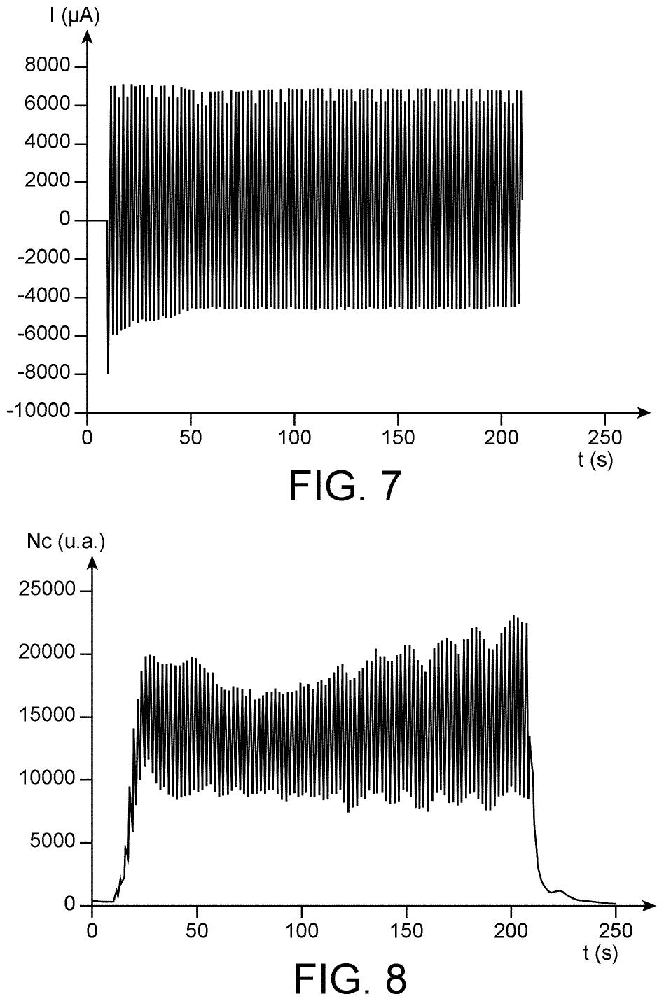

In FIGS. 5 and 7, the y-axis corresponds to the intensity, denoted I and expressed in milliamperes (mA) in FIG. 5 and in microamperes (μA) in FIG. 7, of the current measured at the working electrode, whereas the x-axis corresponds to time, denoted t and expressed in seconds (s).

In FIGS. 4, 6, 8 to 12, the y-axis corresponds to the number of counts emitted, denoted N_{C} and expressed in arbitrary units (u.a.), whereas the x-axis corresponds to the time, denoted t and expressed in seconds (s).

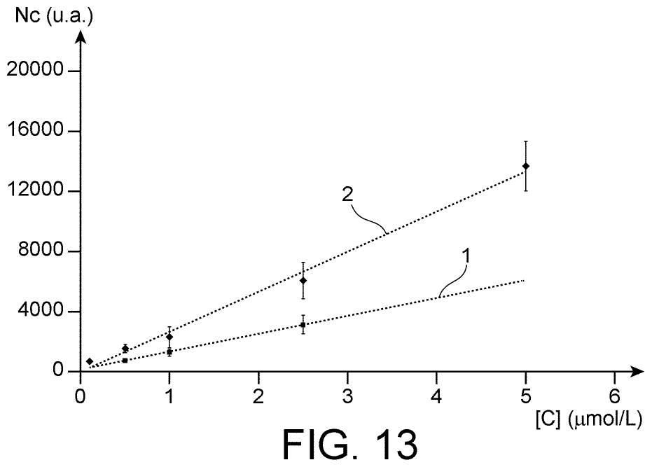

In FIG. 13, the y-axis corresponds to the number of counts emitted, denoted N_{C} and expressed in arbitrary unit (u.a.), whereas the x-axis corresponds to the concentration of skatole, denoted [C] and expressed in micromoles/L (μmol/L).

### DETAILED DESCRIPTION OF PARTICULAR EMBODIMENTS

The experiments which are described below are performed using:
either organic extracts of samples of pig adipose tissue, which is either contaminated with skatole or not;
or solutions referred to as “standard solutions” in the following and comprising synthetic skatole in acetonitrile.

### I—Preparation of the Organic Extracts of Samples of Pig Adipose Tissues and of the Standard Solutions

I.1—Organic Extracts:

The organic extracts of samples of pig adipose tissues, which are either contaminated with skatole or not, are prepared for each extract according to the following operating protocol.

A sample of adipose tissue from a pig, cut finely, is placed in a glass beaker and brought to a temperature of 120° C. for 1 hour. During this heating, the beaker is not covered to allow the water vapour to escape. The size of the sample of adipose tissue is selected such that at least 5 mL of melted fat is collected at the end of this step.

Then, 5 mL of this melted fat is transferred into a test tube to which 5 mL acetonitrile (purity: 99.8%) are added. The test tube is closed, then the mixture is brought to a temperature of 70° C. for 10 minutes. A solution is obtained which is agitated by means of a vortex stirrer for 5 minutes, then centrifuged at 4000 rpm for 15 minutes to obtain a fatty phase and a solvent phase.

The tube is then placed in a freezer at 18° C. for at least 2 hours to freeze the fatty phase. The solvent phase is recovered and transferred to a new test tube.

To this phase, 100 mg of magnesium sulphate are added to finish drying it, then 0.385 mg tetrabutylammonium hexafluorophosphate (TBAHFF) are added to give it a ground salt concentration of 0.1 mol/L.

The extracts prepared in this way are kept in the refrigerator until used.

I.2—Standard Solutions:

The standard solutions are prepared by dissolving, with stirring, synthetic skatole in acetonitrile in a concentration ranging from 0.1 μmol/L to 5 μmol/L, then adding TBAHFF to the resulting solutions to give them a ground salt concentration of 0.1 mol/L.

### II—Detection of Skatole

II.1—Experimental Apparatus:

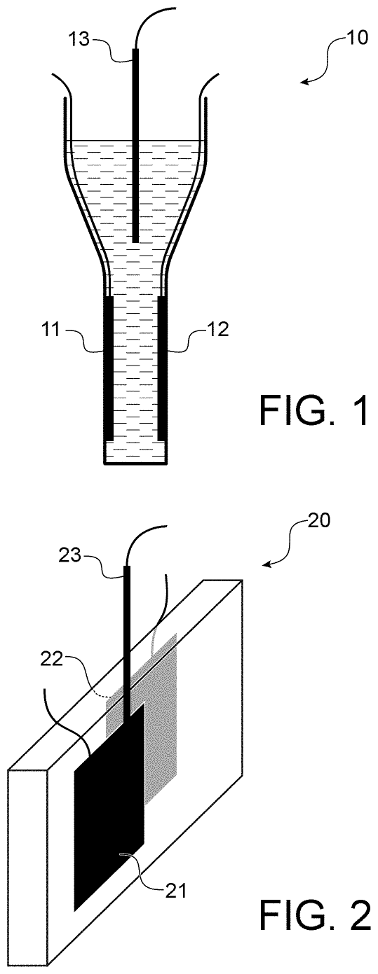

The ECL reactions are performed by using the two electrochemical cells, denoted 10 and 20 respectively, which are illustrated schematically in FIGS. 1 and 2, and a PalmSens4™ potentiostat (PALMSENS).

The cell 10 comprises a single use UV/visible spectroscopy cuvette, made of polystyrene, the lower part of which has a quadrangular cross-section and in which two electrodes 11 and 12 made of boron-doped diamond, used respectively as the working electrodes and counter electrode, are positioned on two opposite walls of this lower part such that these electrodes face one another. The electrodes 11 and 12 have a surface area of 1 cm².

The cell 20 itself comprises a parallelepiped-shaped cuvette in which two boron-doped diamond electrodes 21 and 22, used respectively as the working electrode and the counter electrode, are positioned on the two opposite walls of this cuvette which have the largest surface area but offset from one another so that these electrodes are arranged parallel to one another but without facing one another. The electrodes 21 and 22 have a surface area of 4 cm².

The cells 10 and 20 are further provided with a platinum wire, respectively 13 and 23, used as a pseudo-reference electrode.

The detection of photons emitted during the ECL reactions is carried out in the wavelength range of 450 nm to 550 nm, by means of a Fluoromax™ 4 or 4P spectrofluorometer (HORIBA JOBIN YVON) which makes it possible to monitor in real time the evolution of the luminescence by means of integrated software.

During the ECL reactions, the electrochemical cells are placed in absolute darkness to limit the background noise of the spectrofluorometer.

II.2—Deprotonation of Organic Acids:

The measurements of skatole in the organic extracts are performed after the deprotonation of the organic acids present in these extracts.

This deprotonation is obtained by adding to each extract sodium hydride (NaH) in the form of a 60% dispersion of NaH in mineral oil (CAS: 7646-69-7), in an amount of 60 mg of this dispersion for 5 mL of extract. During this addition, bubbling is observed, corresponding to a release of dihydrogen. The extract is then left to rest for 15 minutes during which a precipitate forms, corresponding to the saponification of organic acid present in the extract. This precipitate is removed.

II.3—Verification of the Possibility of Performing the ECL Reaction According to Different Electrochemical Protocols:

As previously indicated, the ECL reaction of skatole which is preferred in the context of the invention is a reaction caused by the application of a cathodic potential to the working electrode of an electrochemical cell to induce the formation of superoxide ions, in an organic extract obtained from a sample of pig adipose tissue, by reducing the dioxygen dissolved in this extract.

This reaction can be broken down into the following steps:
application of a cathodic potential:
(1) formation of superoxide ions by reduction of dissolved dioxygen:

O₂+e⁻↔O₂⁻.
(2) formation of the conjugate base of skatole in the presence of superoxide ions:

3-MIH+O₂⁻.→3-MI⁻+HO₂.
(3) formation of hydroperoxyl radicals:

O₂⁻.+HO₂.→O₂+HO₂⁻
application of an anodic potential:
(4) oxidation of skatole:

3-MI⁻-e⁻→3-MI.

with:

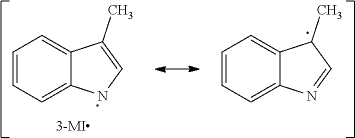

(5) radical coupling of oxidised skatole with HO involving the formation of an intermediate compound comprising a 1,2-dioxetane group followed by N-(2-acetylphenyl)formamide in an excited state:

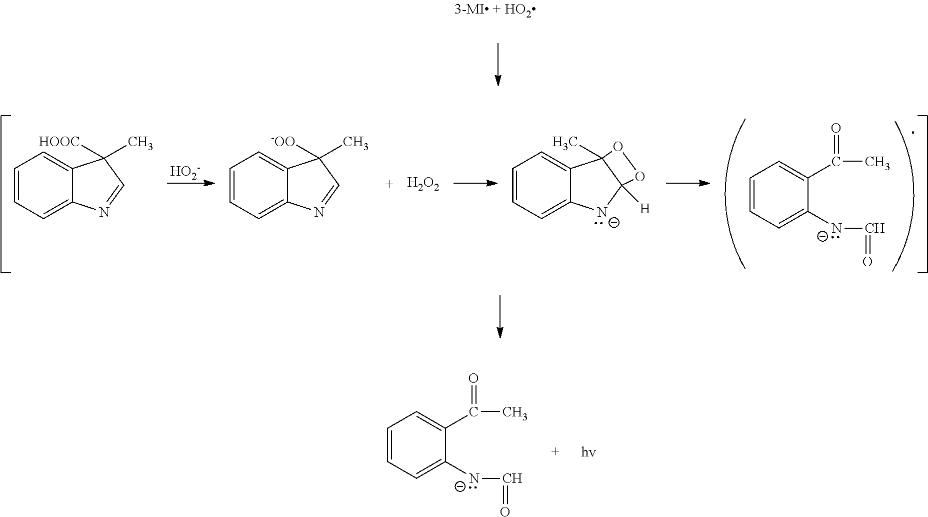

To obtain this reaction, three electrochemical protocols are tested:
a potential sweep (cyclic voltammetry);
a potential step (chronoamperometry); and
a repeated application of alternating cathodic and anodic voltage pulses.

Potential Sweep:

For the potential sweep, the following are used:
an organic extract of a sample of a pig adipose tissue not contaminated with skatole to which synthetic skatole has been added in a concentration of 5 μmol/L, and
the electrochemical cell 10 shown in FIG. 1.

After introducing the organic extract into the electrochemical cell, a potential sweep from 0 to −2 V followed by a potential sweep from −2 V to +0.8 V (versus the reference electrode 13) are applied to the working electrode 11 of this cell with a sweep speed of 50 mV/s.

The luminescence measurement is started from the beginning of the potential sweep.

The thus obtained voltammogram and the thus measured luminescence are illustrated in FIGS. 3 and 4 respectively.

As shown in FIG. 4, a luminescence peak is observed in the anodic potential sweep zone, which can be related to the presence of skatole in the organic extract and the amplitude of which can be connected to the quantity of skatole present in this extract.

Potential Step:

For the potential step, the following are used:
an organic extract of a sample of pig adipose tissue not contaminated with skatole to which synthetic skatole has been added in a concentration of 5 μmol/L, and
the electrochemical cell 20 shown in FIG. 2.

Having introduced the organic extract into the electrochemical cell, a potential of −2 V (versus the reference electrode 23) for 30 seconds, followed by a potential of +1 V (versus the reference electrode 23) for a further 30 seconds are applied to the working electrode 21 of this cell.

Here also, the luminescence measurement is started from the beginning of the application of potential to the working electrode.

The thus obtained chronoamperogram and the thus measured luminescence are illustrated in FIGS. 5 and 6 respectively.

As shown in FIG. 6, an intense peak of luminescence is observed during the passage from the first potential to the second potential, this peak being able, there also, to be related to the presence of skatole in the organic extract and the amplitude of which may be related to the quantity of skatole present in this extract.

Pulse Regime:

The test which consists of applying to the working electrode a repetition of alternating cathodic and anodic voltage pulses is performed by using:
a standard solution of 5 μmol/L skatole, and
the electrochemical cell 20 shown in FIG. 2.

After introducing the standard solution into the electrochemical cell, a potential of −2 V (versus the reference electrode 23) followed by a potential of +1 V (versus the reference electrode 23) are applied to the working electrode 21 of this cell in a repeated manner at a frequency of 1 Hz.

Here too, the measurement of the luminescence is started from the beginning of the application of potential to the working electrode.

The thus recorded current response of the working electrode and the thus measured luminescence are illustrated in FIGS. 7 and 8 respectively.

As shown in FIG. 8, the emission of a variable luminescence signal but with an average intensity of around 14 000 counts is observed throughout the duration of the application of the pulse regime.

Another test is carried out in the same experimental conditions except that a frequency of 5 Hz is used instead of 1 Hz. A similar ECL response is obtained but with an average luminescence intensity of about 8 000 counts, that is to say weaker than the preceding one.

II.4—Verification of the Specific Nature of the Detection of Skatole:

Two experiments are carried out, namely:
a first experiment which consists of subjecting a statistically significant series of organic extracts from samples of pig adipose tissues not contaminated with skatole (the absence of skatole in these extracts having been verified by gas chromatography coupled with mass spectrometry or GC/MS, which makes it possible to dose skatole to a concentration of 0.03 μg/g fat), in an ECL reaction and to measure the luminescence emitted during this reaction;
a second experiment which consists of subjecting to an ECL reaction, on the one hand, a standard solution of 5 μmol/L skatole, and, on the other hand, solutions comprising 5 μmol/L indole and 0.1 mol/L TBAHFF in acetonitrile, in the same operating conditions and measure the luminescence emitted during this reaction.

In this regard, it should be noted that skatole and indole only differ structurally from one another in that the pyrrole cycle of skatole bears a methyl group contrary to the pyrrole cycle of indole.

The first experiment is performed in the electrochemical cell 10 shown in FIG. 1 and by applying a potential step, as described in point II.3 above.

The second experiment is performed in the electrochemical cell 20 shown in FIG. 2 and by applying a potential sweep, as also described in point II.3 above.

No significant luminescence signal is detected during the first experiment, the measured luminescence being of the type shown in FIG. 12.

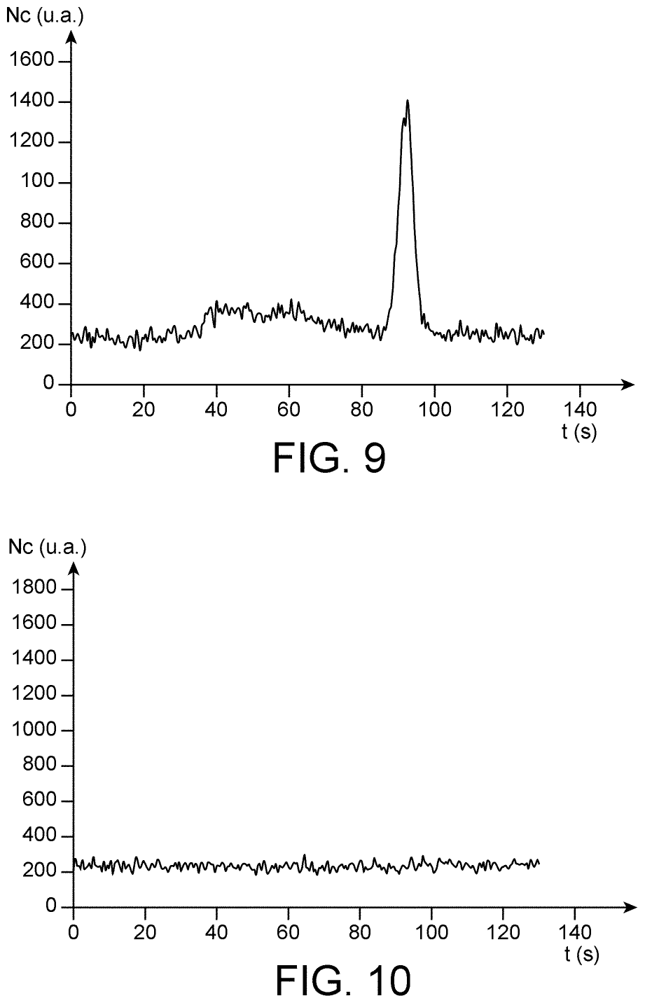

As shown in FIGS. 9 and 10 which represent examples of the luminescence measured during the second experiment for a standard solution of skatole and a solution of indole respectively, an intense luminescence signal is observed for the standard solution of skatole, whereas no significant luminescence signal is observed for the indole solution, thus confirming the very specific nature of the detection of skatole by the method of the invention.

II.5—Blind Skatole Detection Tests:

Skatole detection tests are performed by subjecting organic extracts of samples of a pig adipose tissue contaminated with skatole (0.27 μg skatole/g adipose tissue) and organic extracts of a pig adipose tissue not contaminated with skatole to an ECL reaction in the electrochemical cell 20 shown in FIG. 2, by applying a potential step as described in point II.3 above and by measuring the luminescence emitted during this reaction.

The contamination and absence of contamination of organic extracts were checked beforehand by GC/MS.

The tests were carried out blind, i.e. the experimenter does not know when subjecting an extract to an ECL reaction whether this extract is contaminated or not with skatole.

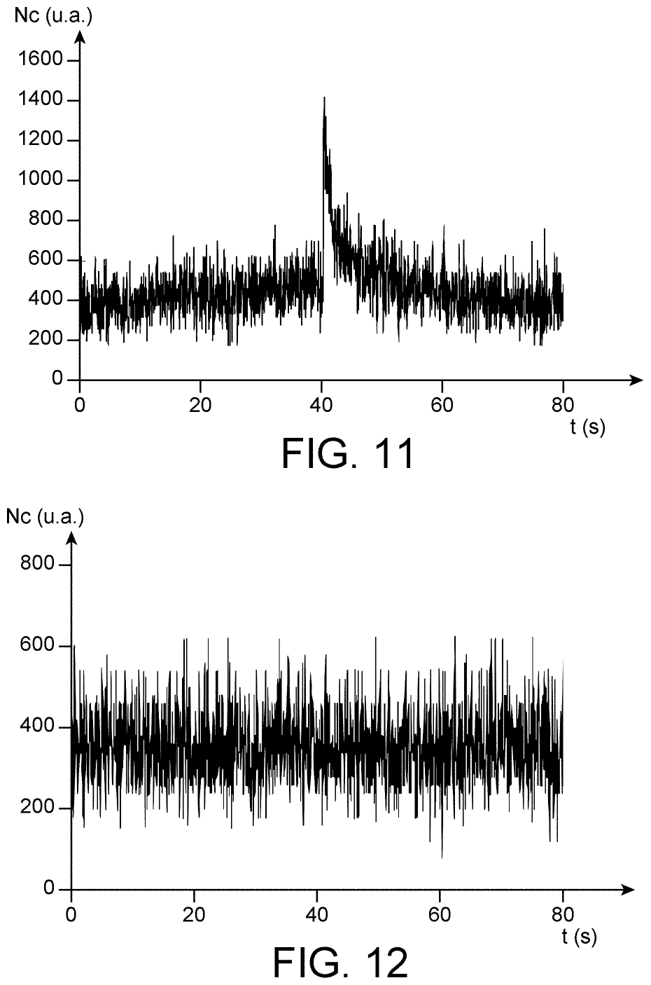

FIG. 11 shows a luminescence signal as typically observed for an organic extract of a sample of pig adipose tissue contaminated with skatole, whereas FIG. 12 shows the absence of a significant luminescence signal as observed for an organic extract of a sample of pig adipose tissue not contaminated with skatole.

II.6—Calibration Curves:

In order to verify the possibility of quantifying the skatole present in an organic extract of pig adipose tissue, calibration curves are produced by subjecting:
on the one hand, standard solutions of skatole comprising 0.1 μmol/L to 5 μmol/L skatole; and
on the other hand, organic extracts of samples of a pig adipose tissue not contaminated with skatole but with the addition of synthetic skatole in an amount of 0.1 μmol/L to 5 μmol/L;
   to an ECL reaction and measuring the maximum intensity of the luminescence emitted during this reaction.

The ECL reaction is performed in the electrochemical cell 10 shown in FIG. 1 and by applying to the working electrode 11 of this cell a potential of −2 V (versus the reference electrode 13) for 30 seconds, then of +1 V (versus the reference electrode 13) for another 30 seconds.

The calibration curves obtained in this way are illustrated in FIG. 13, curve 1 corresponding to the organic extracts with added synthetic skatole and the curve 2 corresponding to standard solutions of skatole.

This figure shows, for the two curves, an almost linear relationship between the amplitude of the peaks of luminescence and the concentration of skatole.

The slope of the curve 1 is approximately twice as low as that of the curve 2, which means that the detection sensitivity of the skatole in the case of organic extracts with added synthetic skatole is about twice as low as in the case of standard solutions of skatole.

This is explained experimentally, first of all, by the fact that the organic extracts with synthetic skatole added reabsorb 30% of the photons emitted (measurement carried out by UV/visible spectroscopy). The loss of the remaining 20% of signals is probably due to the fact that the organic extracts with synthetic skatole added have a higher viscosity than the standard solutions of synthetic skatole and therefore a lower ionic mobility which slows down the diffusion processes governing the electrochemical processes.

However, these two phenomena (reabsorption of photons and increase in viscosity) can be easily corrected from, on the one hand, the absorption coefficient of the organic extract analysed at the wavelength of interest (use of the average value which is not likely to vary significantly from one organic extract to another, or where necessary the measurement performed in parallel on each organic extract), and on the other hand diffusion coefficients of the ionic species involved in the analysed organic extract that have been evaluated previously.

### CITED REFERENCES

K. Verplanken and al, Journal of Chromatography A 2016, 1462, 124-133
M. P. Aguilar-Caballos and al, Analytica Chimica Acta 2002, 460, 271-277
WO-A-83/00928
EP-A-2 966 441
X. Liu and al, Meat Science 2016, 116, 133-139
T. Okajima and T. Ohsaka, Journal of Electroanalytical Chemistry 2002, 523, 34-39
U.S. Pat. No. 9,121,107

What is claimed is:

1. A method for detecting a presence of skatole in a sample of a pig adipose tissue, comprising at least the steps of:
a) subjecting an organic extract of the sample of the adipose tissue to an electrochemiluminescence reaction;
b) measuring an intensity of a luminescence produced during step a) and, if the measured luminescence intensity exceeds a predetermined threshold value, deducing the presence of skatole in the sample of the adipose tissue.

2. The method of claim 1, wherein the organic extract comprises an aprotic organic solvent, less than 1 wt. % water, and a ground salt.

3. The method of claim 2, which comprises, prior to step a), preparation of the organic extract, said preparation comprising the steps of:
i) separating the fat of the sample of the adipose tissue from the non-fatty elements of the sample of the adipose tissue;
ii) dehydrating the fat of the sample of the adipose tissue;
iii) dissolving the fat obtained at the end of step ii) in the aprotic organic solvent to obtain an organic solution and heating the organic solution;
iv) degreasing the organic solution obtained at the end of step iii); and
v) adding the ground salt to the organic solution obtained at the end of step iv), the ground salt being an anhydrous ground salt;
and wherein steps i) and ii) are carried out successively or simultaneously.

4. The method of claim 3, wherein steps i) and ii) are carried out simultaneously.

5. The method of claim 4, wherein steps i) and ii) comprise heating the sample of the adipose tissue to a temperature of 100° C. to 150° C. in an open container, or to a temperature lower than 100° C. with a vacuum drawing.

6. The method of claim 2, wherein the organic solvent is acetonitrile, dimethylsulfoxide, propylene carbonate or γ-butyrolactone.

7. The method of claim 2, wherein the heating of step iii) is carried out in a closed container, at a temperature of 50° C. to 80° C., for 10 minutes to 30 minutes and with stirring.

8. The method of claim 2, wherein step iv) comprises centrifugating the organic solution obtained at the end of step iii), then fixing the fatty part of the organic solution by maintaining the organic solution at a temperature lower than or equal to 4° C. but higher than a solidification temperature of the aprotic organic solvent, and removing the fatty part that has been fixed.

9. The method of claim 2, wherein the ground salt is a tetraalkylammonium tetrafluoroborate, hexafluorophosphate or perchlorate wherein the alkyl group comprises 1 to 4 carbon atoms.

10. The method of claim 2, wherein the organic extract comprises 0.01 mol/L to 1 mol/L of the ground salt.

11. The method of claim 2, wherein the organic extract further comprises a strong base.

12. The method of claim 11, wherein the preparation of the organic extract further comprises a step vi) consisting of adding the strong base to the organic solution obtained at the end of step v).

13. The method of claim 1, wherein step a) comprises introducing the organic extract in an electrochemical cell comprising at least one working electrode and one counter-electrode, and applying a cathodic potential followed by an anodic potential to the working electrode.

14. The method of claim 13, wherein the application of the cathodic potential followed by the anodic potential to the working electrode comprises a potential sweep, a potential jump or a series of alternate cathodic and anodic potential pulses.

15. The method of claim 13, wherein the working electrode and the counter-electrode are made of doped diamond.

16. The method of claim 13, wherein the electrochemical cell is for single use.

17. The method of claim 1, further comprising a quantification of the skatole present in the organic extract, the quantification comprising comparing a maximum of the luminescence intensity measured during step b) with a calibration curve.

18. A method for sorting carcasses of whole male pigs, comprising an implementation of a method of claim 1.
